(19) United States
(12) Patent Application Publication
(10) Pub. No.: US 2023/0083459 A1
(43) Pub. Date: Mar. 16, 2023

(54) METHOD OF PRODUCING BIO-COMPATIBLE CROSS LINKED POLYMERS FOR SPRAY APPLICATIONS

(71) Applicants: PALO ALTO RESEARCH CENTER INCORPORATED, Palo Alto, CA (US); XEROX CORPORATION, Norwalk, CT (US)

(72) Inventors: Ravi Neelakantan, San Diego, CA (US); Alexander Rousina-Webb, Hamilton (CA)

(21) Appl. No.: 17/474,417

(22) Filed: Sep. 14, 2021

(51) Int. Cl. B29B 7/00; B29B 7/24

(52) U.S. Cl. CPC B29B 7/002; B29B 7/24; A61F 2013/0011

(57) ABSTRACT

A system has a first fluid dispenser containing a biocompatible polymer, a second fluid dispenser containing a biocompatible curing agent selected to form a highly viscous mixture with the biocompatible polymer, a pair of diverging surfaces having a contact point, the first fluid dispenser and the second fluid dispenser positioned to dispense a first fluid and a second fluid at the contact point, and an actuator connected to the first fluid dispenser, the second fluid dispenser and the pair of diverging surfaces, the actuator configured to cause the first fluid dispenser and the second fluid dispenser to dispense the fluids at the contact point, and to cause the diverging surfaces to move through the contact point and then diverge, causing the mixture to form filaments until the filaments break up to form a spray. A method of forming a spray includes dispensing a biocompatible polymer solution and a curing agent onto a pair of diverging surfaces to form a mixture, moving the two diverging surfaces counter to each other to cause the mixture to form filaments between the two diverging surfaces as the diverging surfaces move away from each other, moving the two diverging surfaces further counter to each other to cause the filaments burst into droplets, and directing the droplets onto a surface. A composition of matter comprising a biocompatible polymer and a biocompatible curing agent.

### TECHNICAL FIELD

This disclosure relates to biologically compatible (bio-compatible) polymers, more particularly to polymers that can crosslink using a bio-compatible curing agent.

### BACKGROUND

Existing spray-on bandages generally consist of low-viscosity solutions composed of dilute and/or low molecular weight polymers dissolved in a volatile solvent. After deposition of the solution, the solute rapidly evaporates leaving a coalesced film covering the wound. Due to the low viscosity and the low surface tension, the fluid forms thin films, making formation of thicker and more robust spray-on bandages improbable.

Ideally, spray-on bandages would be thicker and more robust, capable of incorporating drugs for slow release, similar to drug patches. This would allow a field-deployable, one-size-fits-all bandaging solution applicable in a fraction of the time needed for traditionally applied bandages.

Conventional atomizers and sprayer typically cannot spray viscous fluids. This limits the scope of potential spray-on bandages to dilute solutions of polymers, typically with a low-to-moderate molecular mass.

This results in a choice between sprayable compositions that cannot result from viscous fluids, resulting in a thin bandage, or using viscous fluids that eliminate the spray-on application.

### SUMMARY

According to aspects illustrated here, there is provided a system having a first fluid dispenser containing a biocompatible polymer, a second fluid dispenser containing a biocompatible curing agent selected to form a highly viscous mixture with the biocompatible polymer, a pair of diverging surfaces having a contact point, the first fluid dispenser and the second fluid dispenser positioned to dispense a first fluid and a second fluid at the contact point, and an actuator connected to the first fluid dispenser, the second fluid dispenser and the pair of diverging surfaces, the actuator configured to cause the first fluid dispenser and the second fluid dispenser to dispense the fluids at the contact point, and to cause the diverging surfaces to move through the contact point and then diverge, causing the mixture to form filaments until the filaments break up to form a spray.

According to aspects illustrate here, there is provided a method of forming a spray including dispensing a biocompatible polymer solution and a curing agent onto a pair of diverging surfaces to form a mixture, moving the two diverging surfaces counter to each other to cause the mixture to form filaments between the two diverging surfaces as the diverging surfaces move away from each other, moving the two diverging surfaces further counter to each other to cause the filaments burst into droplets, and directing the droplets onto a surface.

According to aspects illustrate here, there is provided a composition of matter comprising a biocompatible polymer and a biocompatible curing agent.

### BRIEF DESCRIPTION OF THE DRAWINGS

FIG. 1 shows an embodiment of a system to spray a viscous, reactive fluid.

FIGS. 2-4 show embodiments of filament extension atomizers with diverging surfaces.

FIG. 5 shows a flowchart of an embodiment of a method of manufacturing a sprayable formulation.

### DETAILED DESCRIPTION OF THE EMBODIMENTS

Generating sprays or mists from highly viscous materials presents several problems, mostly due to the ‘thickness,’ or resistance to flow of the material. The particles of the materials tend to have internal friction between layers of the fluid that flow at different rates, making them ‘sticky’ and hard to separate from other particles of the material. The terms “highly viscous” or “high viscosity” mean a viscosity of over 1 mPa-s (milliPascal-second).

The filament extension atomizer (FEA) technology developed at PARC emerged as a solution to this problem. FEA enables spraying of highly viscous formulations. Thin, low viscosity fluids, with or without a propellant, can easily be sprayed using pump or trigger sprayers. These methods do not work with higher viscosity fluids, but the FEA has had success in spraying these liquids.

Generally, FEA systems have diverging surfaces, such as a pair of counter-rotating rollers. The two surfaces come into contact and then move away from each other. Typically, a fluid is applied to one or both of the surfaces. The surfaces move towards each other and come into contact, or near contact, then diverge from each other. During contact, the fluid sticks to the surfaces, and then as them move away from each other, the fluid forms filaments that stretch between the surfaces until the strain causes the filament to burst into droplets and form the spray.

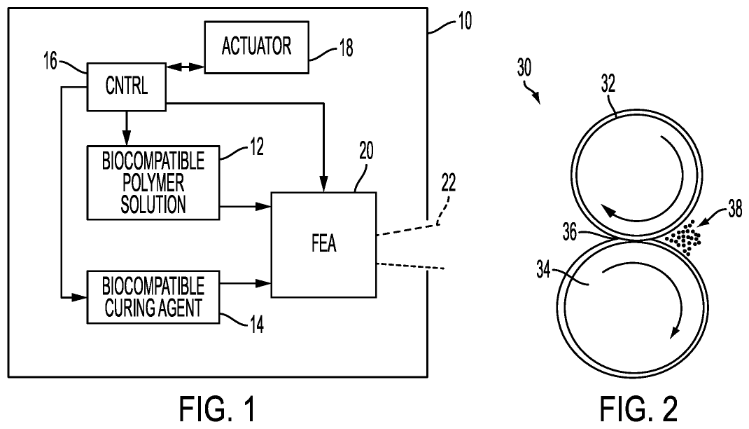

FIG. 1 shows an embodiment of a system that uses an FEA atomizer to spray a viscous material and curing agent onto a surface. The atomizer 10 may be contained in a housing that contains all of the components. The housing may have an opening that allows the resulting spray to be applied to a surface. Unlike the typical FEA system, this system employs two fluids, not just one. In this case, the two fluids form a two-part reactive system, where one of the fluids has a curing agent that reacts to cause the other fluid to cure at the surface. The term “curing” as used here means any agent that causes the polymers to cross-link.

This system has one fluid dispenser containing a biocompatible polymer solution, and another fluid dispenser containing a biocompatible curing agent. As used here, the term “biocompatible” means any material not harmful to living tissue. The polymers used comprised biocompatible polymers, and the curing agent comprises a biocompatible curing agent. In some embodiments, the biocompatible polymer may comprise a biopolymer. As used here, “biopolymer” means a polymeric substance occurring in living organisms, such as polypeptides made up of amino acids, polysaccharides that include starches and sugars, and polynucleotides like DNA and RNA.

In the FEA component, the surfaces will be referred to as “diverging,” which means that the surfaces move away from each other at some point in their movement. The surfaces in this case will come into contact or near-contact and then move away from each other. As used here “near-contact” means that the surfaces come into close enough contact that the material sticks to both surface regardless of whether the materials were applied to one or both surfaces. The term “contact” will refer to both contact and near-contact for purposes of this discussion.

The materials may both be applied to one surface and then the other surface comes into contact with them at what is called the nip, the region where the two rollers are in contact or in the closest contact with each other. Each material may be applied separately, one to each roller, and then they combine at the nip to form a mixture. The material may also be applied into the nip directly, essentially applying both materials at the same time.

In FIG. 1, the system has a dispenser 12 that contains a biocompatible polymer solution, a dispenser 14 that contains a biocompatible curing agent. The biocompatible biopolymer solution may comprise the biocompatible polymer by itself or with other materials, such as a buffering material, coagulants, pain killers, antibacterial agents, and other drugs. The buffering material may include a carbonate material, used to maintain the pH in the range of 7 to 10, but of at least 7. The biocompatible polymer may comprise one of several biocompatible polymers including, but not limited to, polyvinyl alcohol (PVA), hydroxyl propyl guar (HPG), hydroxyl propyl methylcellulose (HPMC), biopolymers, and a polymer having a 1,3-diol groups.

The biocompatible curing agent may comprise boric acid. Other biocompatible curing agents may be used, such as thiol-aldehyde combinations may result in weakly cross-linked structures. However, these typically require rarer aldehyde-containing sugars. These may also require some catalysis with a strong acid. Glutaraldehyde can also act as a cross-linker, but only cross-links at very high temperatures with an acid. The acid may result in a non-biocompatible result, so these may not be useful in applications involving interactions with human tissue.

The system 10 may also include an actuator 18 that activates the FEA component and the dispensers. The actuator could be something as simple as a button, pump or trigger that changes the pressure inside the system and causes the rollers to rotate. However, more than likely, some sort of controller will control the dispensers and the rollers. The controller 16 may comprise a microcontroller, a general purpose processor, a field-programmable gate array (FPGA), and application specific integrated circuit (ASIC), among many others. When the FEA component 20 generates the spray 22, it exits the housing to reach a surface.

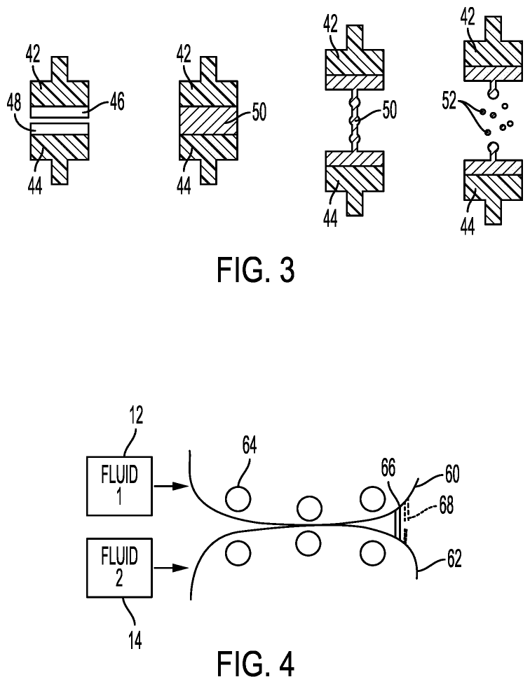

The FEA component may take many different forms. FIG. 2 shows an embodiment 30 of the FEA component that comprises two counter-rotating rollers 32 and 34. The region where the two rollers come into contact, the nip 36, may cause the two materials to mix, or the two materials may mix within a secondary dispenser just prior to being sprayed onto the FEA component. As the rollers move away from each other, the filaments break up into droplets such as 38.

FIG. 3 shows an alternative embodiment of an FEA component. In this embodiment, the two diverging surface comprise a piston 42 and either another piston or a fixed surface 44. Either surface 46 or 48 could move and the other one is fixed, or they both move. As discussed above, either or both surfaces could be coated by a combination of the two fluids, or each fluid separately. When the surfaces come together, they form the mixture 50. As the two surfaces diverge, the mixture 50 forms a filament, that eventually break apart and form droplets such as 52.

FIG. 4 shows another alternative of an FEA component, using belts as the diverging surfaces. The dispensers would each dispense their fluids on the two belts 60 and 62. Rollers such as 64 move the belt into contact with each other and then away from each other. This forms the filaments such as 66 and then the droplets 68.

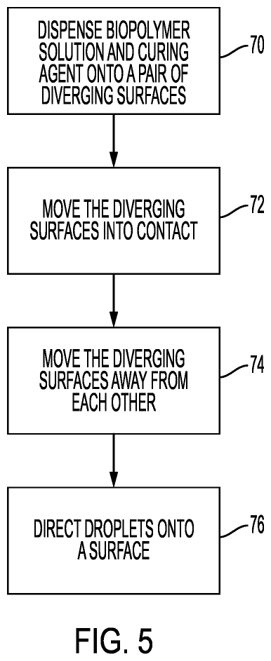

FIG. 5 shows a flowchart of an embodiment of a method of generating a spray-on, curable formulation. At 70, the process dispenses the biopolymer solution and the curing agent onto a pair of diverging surfaces. After the material is dispensed, the diverging surfaces move into contact that allow the materials to mix, or the materials mix just before the contact region of the diverging surfaces at 72. The concentration of the two fluids should be managed to produce the desired gel. If the mixture has too high of a concentration of boric acid, the material turns into a hard, rubbery material. If the mixture has too high of a concentration of boric acid, the material would leave an uncured gel. The process then moves the diverging surfaces away from each other, which then cause the filaments to form and break into droplets at 74. The droplets are then directed onto a surface at 76, such as a wound or skin. The directing may take the form of just pointing the atomizer towards the surface, or there may be an air source to provide an air stream to direct the droplets in a predetermined direction.

In this manner, one can spray more viscous materials that can hold other agents on a spray site, and the introduction of the curing agent allows the viscous material to cure and form a thick gel.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the embodiments.

What is claimed is:

1. A system, comprising:
a first fluid dispenser containing a biocompatible polymer;
a second fluid dispenser containing a biocompatible curing agent selected to form a highly viscous mixture with the biocompatible polymer;
a pair of diverging surfaces having a contact point, the first fluid dispenser and the second fluid dispenser positioned to dispense a first fluid and a second fluid from the contact point; and
an actuator connected to the first fluid dispenser, the second fluid dispenser and the pair of diverging surfaces, the actuator configured to cause the first fluid dispenser and the second fluid dispenser to dispense the fluids at the contact point, and to cause the diverging surfaces to move through the contact point and then diverge, causing the mixture to form filaments until the filaments break up to form a spray.

2. The system as claimed in claim 1, wherein the diverging surfaces comprise one of a pair of counter-rotating rollers, a piston and opposing surface, or opposing belts.

3. The system as claimed in claim 1, wherein the first fluid dispenser also contains a buffer mixed with the biocompatible polymer.

4. The system as claimed in claim 1, wherein the system is contained in a housing.

5. The system as claimed in claim 1, wherein at least one of the first fluid dispenser and the second fluid dispenser contains one or more additional agents.

6. The system as claimed in claim 1, wherein the additional agents include coagulants, pain killers, antibacterial agents, and other drugs.

7. A method of forming a spray, comprising:
dispensing a biocompatible polymer solution and a curing agent onto a pair of diverging surfaces to form a mixture;
moving the two diverging surfaces counter to each other to cause the mixture to form filaments between the two diverging surfaces as the diverging surfaces move away from each other;
moving the two diverging surfaces further counter to each other to cause the filaments burst into droplets; and
directing the droplets onto a surface.

8. The method as claimed in claim 7, wherein the biocompatible polymer comprises a 1,3-diol polymer.

9. The method as claimed in claim 7, wherein the biocompatible polymer comprises a biopolymer.

10. The method as claimed in claim 9, wherein the biopolymer comprises one of polypeptides and polysaccharides.

11. The method as claimed in claim 7, wherein the biocompatible curing agent comprises boric acid.

12. The method as claimed in claim 7, wherein the biocompatible polymer solution further comprises a biocompatible polymer and a buffer.

13. The method as claimed in claim 7, wherein the biocompatible polymer solution comprises one of polyvinyl alcohol, hydroxyl propyl guar, and hydroxyl methylcellulose.

14. The method as claimed in claim 7, wherein the biocompatible polymer solution has a pH of at least 7.

15. The method as claimed in claim 7, wherein at least one of the first fluid dispenser and the second fluid dispenser contains one or more additional agents.

16. A composition of matter, comprising a biocompatible polymer and a biocompatible curing agent.

17. The composition of matter as claimed in claim 15, wherein the biocompatible polymer comprises a 1,3-diol polymer.

18. The composition of matter as claimed in claim 15, wherein the biocompatible polymer comprises a biopolymer.

19. The composition of matter as claimed in claim 15, wherein the biocompatible curing agent comprises boric acid.

20. The composition of matter as claimed in claim 15, wherein the biocompatible polymer comprises one of polyvinyl alcohol, hydroxyl propyl guar, and hydroxyl propyl methylcellulose.

21. The composition of matter as claimed in claim 16, further comprising one or more of coagulants, pain killers, antibacterial agents, and other drugs.
